(19) United States
(12) Patent Application Publication
(10) Pub. No.: US 2024/0374677 A1
(43) Pub. Date: Nov. 14, 2024

(54) VARIOUS COMPOUNDS FOR THE TREATMENT OF NEUROFIBROMATOSIS TYPE 1

(71) Applicant: PUSAN NATIONAL UNIVERSITY INDUSTRY-UNIVERSITY COOPERATION FOUNDATION, Geumjeong-gu Busan (KR)

(72) Inventors: Su Jung PARK, Gyeongsangnam-do (KR); Han Sol JUNG, Gyeongsangnam-do (KR)

(21) Appl. No.: 18/599,835

(22) Filed: Mar. 8, 2024

(30) Foreign Application Priority Data
Mar. 8, 2023 (KR) 10-2023-0030472
Mar. 6, 2024 (KR) 10-2024-0032094

(51) Int. Cl. A61K 38/12; A61K 31/166; A61K 31/167; A61K 31/22; A61K 31/485; A61P 25/00

(52) U.S. Cl. CPC A61K 38/12; A61K 31/166; A61K 31/167; A61K 31/22; A61K 31/485; A61P 25/00

(57) ABSTRACT

Provided are various compounds for the treatment of neurofibromatosis type 1. The compounds of the present disclosure show excellent effects of increasing NF1 protein and inhibiting cell proliferation and protein degradation compared to a control group, and thus may be usefully used as various applications including a therapeutic agent for neurofibromatosis type 1, an anticancer agent, and a therapeutic agent for multiple myeloma and lymphoma.

### CROSS-REFERENCE TO RELATED APPLICATIONS

This application is based on and claims priority from Korean Patent Application No. 10-2023-0030472, filed on Mar. 8, 2023, and Korean Patent Application No. 10-2024-0032094, filed on Mar. 6, 2024, with the Korean Intellectual Property Office, the disclosure of which is incorporated herein in its entirety by reference.

### TECHNICAL FIELD

The present disclosure relates to various compounds for the treatment of neurofibromatosis type 1.

### BACKGROUND

Neurofibromatosis type 1 is a progressive disease caused by mutations or defects in a gene encoding a neurofibromin protein and a rare genetic disease which is usually diagnosed in infancy and has the incidence of approximately 1 of 3,500 people. The neurofibromatosis has three types, and the most common type 1 is called Recklinghausen disease and is characterized by multiple neurofibroma, cafe-au-lait spots, and Lisch nodules on the iris.

As a therapeutic agent for neurofibromatosis type 1, selumetinib was approved by the U.S. FDA in 2020 as the first therapeutic agent for plexiform neurofibromatosis in NF1 patients. However, in the case of selumetinib, there is a problem that the possibility of recurrence is very high with drug discontinuation. In addition, most conventional therapeutic agents, including selumetinib, focus only on proteins related to an RAS signaling pathway, so that the utility is limited compared to various symptoms of NF1. Therefore, the development of novel therapeutic agents is required.

Accordingly, the present inventors established a strategy to improve the symptoms of NF1 haploinsufficiency, which occurred due to a quantitative decrease in functional NF1 protein (neurofibromin), by inducing a quantitative increase in the NF1 protein, discovered natural substances for inducing an increase in NF1 protein (neurofibromin) to develop an efficient NF1 therapeutic agent, and then completed the present disclosure.

Meanwhile, it is expected that recent NF1-targeted therapeutic agents may be used in combination to treat various other cancers.

### SUMMARY

The present disclosure has been made in an effort to provide a pharmaceutical composition for treating neurofibromatosis type 1.

In addition, the present disclosure has also been made in an effort to provide a method for treating neurofibromatosis type 1.

In addition, the present disclosure has also been made in an effort to provide a pharmaceutical composition for preventing or treating cancer.

In addition, the present disclosure has also been made in an effort to provide a method for preventing or treating cancer.

An exemplary embodiment of the present disclosure provides a pharmaceutical composition for treating neurofibromatosis type 1.

In addition, another exemplary embodiment of the present disclosure provides a method for treating neurofibromatosis type 1.

In addition, yet another exemplary embodiment of the present disclosure provides a pharmaceutical composition for preventing or treating cancer.

In addition, still another exemplary embodiment of the present disclosure provides a method for preventing or treating cancer.

According to the exemplary embodiments of the present disclosure, compounds have the effect of improving the symptoms of NF1 haploinsufficiency, which occurs due to a quantitative decrease in NF1 protein (neurofibromin) by inducing a quantitative increase in NF1 protein. In addition, the compounds according to the present disclosure have the effect of inhibiting protein degradation (or increasing synthesis). Therefore, the compounds according to the present disclosure may be used variously as a therapeutic agent for neurofibromatosis type 1, an anticancer agent, and a therapeutic agent for multiple myeloma and lymphoma

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram illustrating three mechanisms of a compound to induce an increase in NF1 protein.

FIG. 2 is a schematic diagram illustrating a process of increasing the synthesis of NF1 by 20-O-acetylingenol-3-angelate.

FIG. 3 is a schematic diagram illustrating a process of inhibiting the degradation of NF1 by (10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate.

FIG. 4 is a schematic diagram illustrating a process of inhibiting the degradation of NF1 by R(−) Apomorphine hydrochloride hemihydrate.

FIG. 5 is a schematic diagram illustrating a process of inhibiting the degradation of NF1 by Thiostrepton.

FIG. 6 is a schematic diagram illustrating a process of inhibiting the degradation of NF1 by Vorinostat.

FIG. 7 is a schematic diagram illustrating an experimental process of Experimental Example 1.

FIG. 8 is a graph showing results of activity analysis (Nano-GloHiBiT Lytic Assay) according to Experimental Example 1.

FIG. 9 is a graph showing results of activity analysis (Nano-GloHiBiT Lytic Assay) after treating compounds according to the present disclosure for 6 hours and 24 hours, respectively.

FIG. 10 is an image showing results of a Western blot test of compounds according to the present disclosure.

FIG. 11 is a graph showing MTT Assay results of compounds according to the present disclosure.

FIG. 12 illustrates analysis results of increased NF1-GRD1 protein by concentration and time of 20-O-acetylingenol-3-angelate through Nano-Glo HiBit assay.

FIG. 13 is an image showing results of a Western blot test of 20-O-acetylingenol-3-angelate.

FIG. 14 is an image showing results of a Western blot test of compounds according to the present disclosure.

FIG. 15 is a graph showing results of MTT Assay of 20-O-acetylingenol-3-angelate on skin cells of NF1 patients.

FIG. 16 is an image showing results of a Western blot test of compounds according to the present disclosure.

FIG. 17 is a graph showing MTT Assay results of R(−) Apomorphine hydrochloride hemihydrate on various cancer cells.

FIG. 18 is an image showing results of a Western blot test of compounds according to the present disclosure.

FIG. 19 is a graph showing MTT Assay results of Thiostrepton.

FIG. 20 is a graph showing MTT Assay results of Vorinostat.

### DETAILED DESCRIPTION

In the following detailed description, reference is made to the accompanying drawing, which forms a part hereof. The illustrative examples described in the detailed description, drawing, and claims are not meant to be limiting. Other examples may be utilized, and other changes may be made, without departing from the spirit or scope of the subject matter presented here.

Terms and words used in the present specification and claims should not be interpreted as being limited to typical or dictionary meanings, but should be interpreted as meanings and concepts which comply with the technical spirit of the present disclosure, based on the principle that the present inventors may appropriately define the concepts of the terms to describe his/her own invention in the best manner. Therefore, the exemplary examples described in the present specification and the configurations illustrated in the drawings are merely the most preferred exemplary embodiment of the present disclosure and are not intended to represent all of the technical ideas of the present disclosure, and thus, it should be understood that various equivalents and modifications capable of replacing the exemplary embodiments at the time of this application.

Hereinafter, the present disclosure will be described in detail.

The present disclosure provides a pharmaceutical composition for treating neurofibromatosis type 1, including at least one selected from the group consisting of the following compounds or a pharmaceutically acceptable salt thereof:
20-O-Acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

Here, the structure of each substance may be as shown in Table below, and for convenience of description, 20-O-acetylingenol-3-angelate may be referred to as PN-NF27, (10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15-hexaen-13-olate may be referred to as PN-NF28, R(−) Apomorphine hydrochloride hemihydrate may be referred to as PN-NF30, Thiostrepton may be referred to as PN-NF38, and Vorinostat may be referred to as PN-NF41.

TABLE 1

| Compound | Structure | Name |
|---|---|---|
| 20-O-acetylingenol-3- angelate | 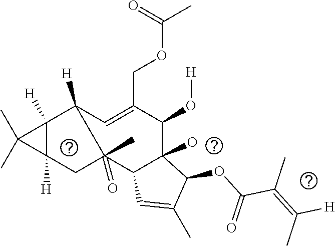 | PN-NF27(27) |
| (10S,14E)-10-acetamido- 3,4,5-trimethoxy-14-[(1- phenylethyl)iminiumyl] tricyclo[9.5.0.0²,^{⁷}] hexadeca- 1(11),2,4,6,12,15- hexaen-13- olate | 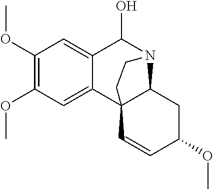 [SMILES: COc1cc2c(cc1OC)[C@@]13C=C[C@@H](OC)C[C@@H]1N(CC3)C2O] | PN-NF28(28) |
| R(−) Apomorphine hydrochloride hemihydrate | 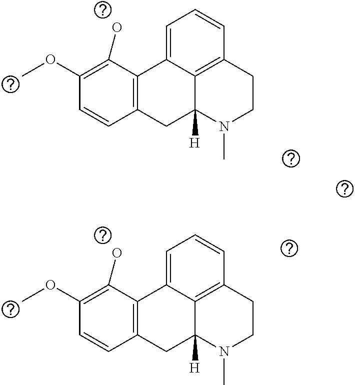 [SMILES: COc1ccc2c(c1O)-c1cccc3c1[C@@H](C2)N(C)CC3 ; COc1ccc2c(c1O)-c1cccc3c1[C@@H](C2)N(C)CC3] | PN-NF30(30) |
| Thiostrepton | [symbol] | PN-NF38(38) |
| Vorinostat | 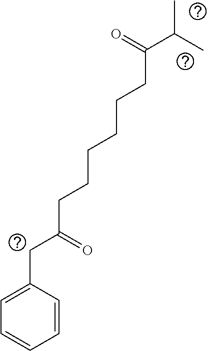 [SMILES: CC(C)C(=O)CCCCCCC(=O)Cc1ccccc1] | PN-NF41(41) |

[symbol]

indicates data missing or illegible when filed

The “20-O-acetylingenol-3-angelate” of the present disclosure, also called PEP008, is a natural product-derived compound and is mainly found in Euphorbiaceae plants. The Euphorbiaceae plants may include Euphorbia peplus, Euphorbia kansui Liou, Euphorbiae Pekinensis, Euphorbiae Lathyridis, and Euphorbiae Fischerianae, but are not limited thereto.

The “(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15-hexaen-13-olate” of the present disclosure is a natural product-derived compound of which use has not been found in any prior art to date, and was distributed by the Korea Chemical Bank.

The “R(−) Apomorphine hydrochloride hemihydrate” of the present disclosure is a natural compound found in Blue lotus (Nymphaea caerulea, Nymphaeaceae plant).

The “Thiostrepton” of the present disclosure is a thiopeptide-based natural compound extracted from the Streptomyces family, such as Streptomyces azureus and Streptomyces laurentii.

The “Vorinostat” of the present disclosure, also called suberoylanilide hydroxamic acid (SAHA), is a natural compound derived from microorganisms. The Vorinostat is an HDAC inhibitor (SAHA) approved by the U.S. Food and Drug Administration (FDA) for a therapeutic agent of lymphoma (2006) and leukemia (2007) and an anticancer agent currently also used to treat lung cancer, brain tumor, and colon cancer. However, the efficacy of Vorinostat in inducing an increase in intracellular NF1 protein has not been reported.

The compounds of the present disclosure may be used in the form of pharmaceutically acceptable salts, and include all salts, hydrates, and solvates prepared by conventional methods.

As the salts, acid addition salts formed with pharmaceutically acceptable free acids are useful. The acid addition salts are prepared by a general method, for example, by dissolving the compound in an excess acid aqueous solution and precipitating the salt using a water-miscible organic solvent, such as methanol, ethanol, acetone or acetonitrile. The same molar amounts of compound and acid or alcohol (e.g., glycol monomethyl ether) in water are heated, and then the mixture may be evaporated and dried, or the precipitated salt may be suction-filtered. At this time, as the free acids, organic acids and inorganic acids may be used. As the inorganic acids, hydrochloric acid, phosphoric acid, sulfuric acid, nitric acid, tartaric acid, etc. may be used, and as the organic acids, methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc. may be used, but the free acids are not limited thereto.

In addition, pharmaceutically acceptable metal salts may be prepared using bases. An alkali metal salt or an alkaline earth metal salt may be obtained, for example, by dissolving the compound in an excess of alkali metal hydroxide or alkaline earth metal hydroxide solution, filtering a non-dissolved compound salt, and then evaporating and drying a filtrate. In this case, the metal salt is pharmaceutically suitable for preparing, particularly, sodium, potassium or calcium salts, but is not limited thereto. Further, silver salts corresponding thereto may be obtained by reacting the alkali metal or alkaline earth metal salt with a suitable silver salt (e.g., silver nitrate).

Pharmaceutically acceptable salts of the compound include salts of acidic or basic groups that may be present in the compound, unless otherwise indicated. For example, the pharmaceutically acceptable salt may include sodium, calcium, or potassium salts of hydroxy groups, and other pharmaceutically acceptable salts of amino groups include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, which may be prepared through a method for preparing a salt known in the art.

In an exemplary embodiment of the present disclosure, the concentration of PN-NF27 may be 0.1 to 10 μM, more preferably 1 μM. If the concentration of PN-NF27 is lower than the above-mentioned range, the effect is minimal, which is not preferable.

In an exemplary embodiment of the present disclosure, the concentration of PN-NF28 may be 0.1 to 10 μM, more preferably 1 μM. If the concentration of PN-NF28 is lower than the above-mentioned range, the effect is minimal, which is not preferable.

In an exemplary embodiment of the present disclosure, the concentration of PN-NF30 may be 1.0 to 10 μM, more preferably 5 μM. If the concentration of PN-NF30 is lower than the above-mentioned range, the effect is minimal, which is not preferable.

In an exemplary embodiment of the present disclosure, the concentration of PN-NF38 may be 1.0 to 10 μM, more preferably 5 μM. If the concentration of PN-NF38 is lower than the above-mentioned range, the effect is minimal, which is not preferable, and if the concentration of PN-NF38 is higher than the above-mentioned range, it is not preferable by exhibiting cytotoxicity.

In an exemplary embodiment of the present disclosure, the concentration of PN-NF41 may be 1.0 to 10 μM, more preferably 5 μM. If the concentration of PN-NF41 is lower than the above-mentioned range, the effect is minimal, which is not preferable, and if the concentration of PN-NF41 is higher than the above-mentioned range, it is not preferable by exhibiting cytotoxicity.

In an exemplary embodiment of the present disclosure, the pharmaceutical composition may induce an increase in neurofibromin protein (NF1 protein).

The present inventors focused on the fact that symptoms of NF1 haploinsufficiency occurred due to a quantitative decrease in functional NF1 protein, found that the compounds of the present disclosure had the effect of inducing an increase in NF1 protein, and a pharmaceutical composition containing the compound may be used as a therapeutic agent for neurofibromatosis type 1, and then completed the present disclosure.

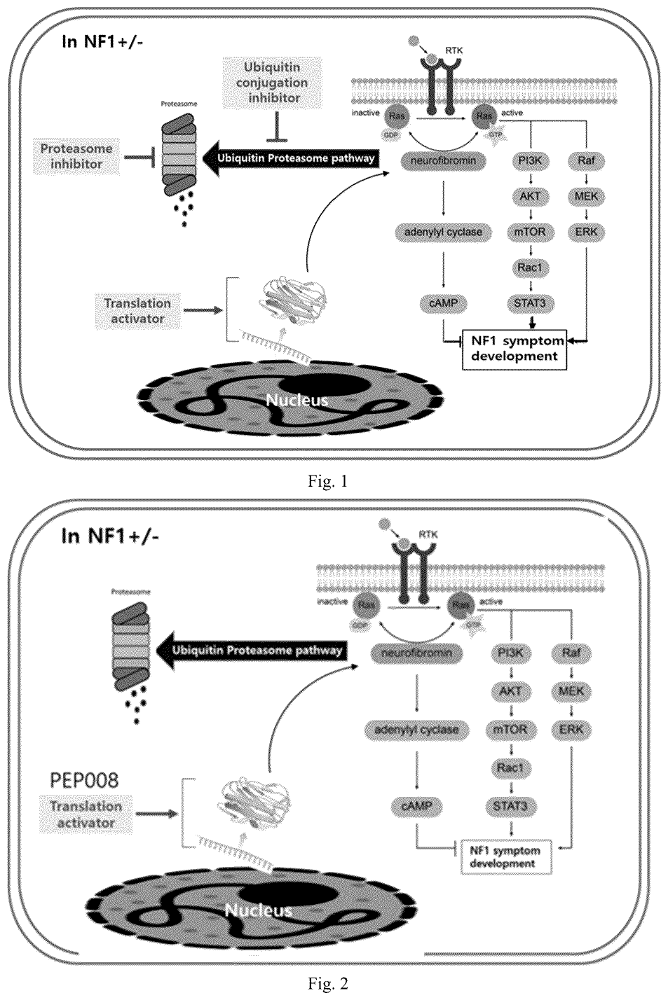

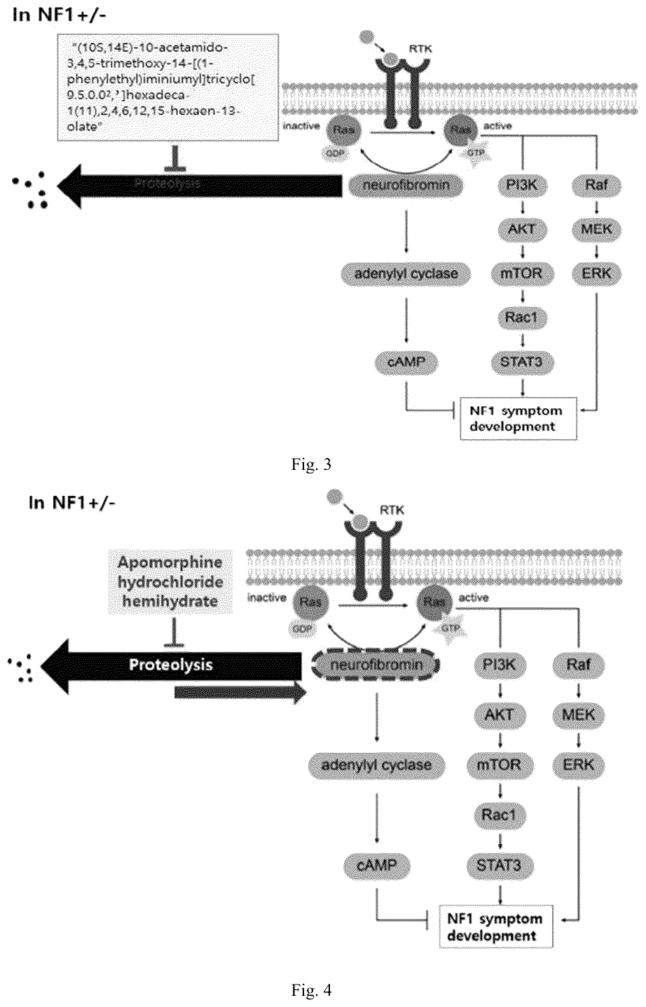

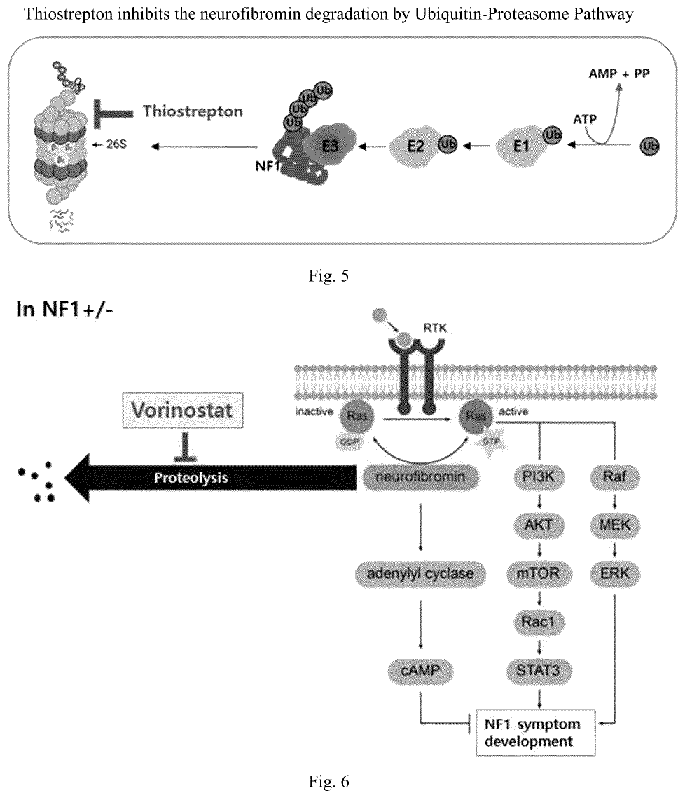

A specific mechanism is as illustrated in FIGS. 1 and 2. An increase in NF1 protein may be induced through three mechanisms (inhibition of ubiquitination pathway, inhibition of proteasome activity, and increase in the activity of NF1 gene transcription/translation) (see FIG. 1). Among the three mechanisms described above, PN-NF27 induces an increase in NF1 protein through a function of increasing the activity of NF1 gene transcription/translation (see FIG. 2), PN-NF28, PN-NF30, and PN-NF41 induce an increase in NF1 protein through a function of inhibiting the ubiquitination pathway (see FIGS. 3, 4, and 6), and PN-NF38 induces an increase in NF1 protein through a function of a proteasome inhibitor (see FIG. 5).

According to another aspect of the present disclosure, the present disclosure provides a method for treating neurofibromatosis type 1 including (a) administering at least one selected from the group consisting of the following compounds into a subject:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

As used in the present disclosure, the “administration” means introducing a predetermined substance into the subject in an appropriate manner.

As used in the present disclosure, the “subject” refers to all animals, such as rats, mice, and livestock, including humans, for the purpose of treating diseases, preferably mammals including humans, and more preferably patients with neurofibromatosis type 1.

According to another aspect of the present disclosure, the present disclosure provides a pharmaceutical composition for preventing or treating cancer, including at least one selected from the group consisting of the following compounds and a pharmaceutically acceptable salt thereof:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

In an exemplary embodiment of the present disclosure, the concentration of PN-NF27 may be 0.5 to 5 μM, more preferably 1 μM. If the concentration of PN-NF27 is lower than the above-mentioned range, the effect is minimal, which is not preferable, and if the concentration of PN-NF27 is higher than the above-mentioned range, it is not preferable by exhibiting cytotoxicity.

In an exemplary embodiment of the present disclosure, the concentration of PN-NF28 may be 0.1 to 10 μM, more preferably 1 μM. If the concentration of PN-NF28 is lower than the above-mentioned range, the effect is minimal, which is not preferable, and if the concentration of PN-NF28 is higher than the above-mentioned range, it is not preferable by exhibiting cytotoxicity.

In an exemplary embodiment of the present disclosure, the concentration of PN-NF30 may be 1.0 to 10 μM, more preferably 5 μM. If the concentration of PN-NF30 is lower than the above-mentioned range, the effect is minimal, which is not preferable, and if the concentration of PN-NF30 is higher than the above-mentioned range, it is not preferable by exhibiting cytotoxicity.

In an exemplary embodiment of the present disclosure, the concentration of PN-NF38 may be 1.0 to 10 μM, more preferably 5 μM. If the concentration of PN-NF38 is lower than the above-mentioned range, the effect is minimal, which is not preferable, and if the concentration of PN-NF38 is higher than the above-mentioned range, it is not preferable by exhibiting cytotoxicity.

In an exemplary embodiment of the present disclosure, the concentration of PN-NF41 may be 1.0 to 10 μM, more preferably 5 μM. If the concentration of PN-NF41 is lower than the above-mentioned range, the effect is minimal, which is not preferable, and if the concentration of PN-NF41 is higher than the above-mentioned range, it is not preferable by exhibiting cytotoxicity.

In an exemplary embodiment of the present disclosure, the cancer may be at least one selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer and bone marrow tumor.

In an exemplary embodiment of the present disclosure, the blood cancer may be multiple myeloma or lymphoma.

In addition to the effect of increasing NF1 protein, the compounds of the present disclosure have the effect of inhibiting protein degradation and inhibiting cell proliferation. Therefore, in addition to being used as the therapeutic agent for neurofibromatosis type 1, the compounds of the present disclosure may be used as a therapeutic agent for cancer, especially a therapeutic agent for multiple myeloma or lymphoma.

According to another aspect of the present disclosure, the present disclosure provides a method for preventing or treating cancer including (a) administering at least one selected from the group consisting of the following compounds into a subject:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

In an exemplary embodiment of the present disclosure, the subject may be a cancer patient.

In addition, the pharmaceutical composition of the present disclosure may be administered orally or parenterally during clinical administration, and when administered parenterally, the pharmaceutical composition may be administered by intraperitoneal injection, intrarectal injection, subcutaneous injection, intravenous injection, intramuscular injection, or intrathoracic injection, and may be used in the form of general pharmaceutical formulations.

The composition of the present disclosure may be preferably formulated as a pharmaceutical composition by further including at least one pharmaceutically acceptable carrier in addition to the above-described active ingredients for administration.

In the composition formulated with a liquid solution, the pharmaceutically acceptable carrier is suitable for sterilization and living bodies and may be used with saline, sterilized water, ringer's solution, buffered saline, albumin injection solution, dextrose solution, maltodextrin solution, glycerol, ethanol, and a mixture of at least one of these ingredients, and if necessary, other general additives such as antioxidants, buffers, bacteriostatic agents, and the like may be added.

In addition, the composition of the present disclosure may be prepared using pharmaceutically suitable and physiologically acceptable adjuvants in addition to the active ingredients. As the adjuvants, solubilizers such as excipients, disintegrants, sweeteners, binders, coating agents, expanding agents, lubricants, slip modifiers, or flavoring agents may be used.

In addition, the composition of the present disclosure may be prepared in injectable formulations such as an aqueous solution, a suspension, and an emulsion, pills, capsules, granules, or tablets by further adding a diluent, a dispersant, a surfactant, a binder, and a lubricant. Furthermore, it may be preferably formulated according to a disease or ingredient by an appropriate method in the art.

The above description just illustrates the technical spirit of the present disclosure and various changes and modifications may be made by those skilled in the art to which the present disclosure pertains without departing from an essential characteristic of the present disclosure. Accordingly, the various exemplary examples disclosed in the present disclosure are not intended to limit the technical spirit but describe the present disclosure and the technical spirit of the present disclosure is not limited by the following exemplary examples. The protective scope of the present disclosure should be construed based on the following claims, and all the techniques in the equivalent scope thereof should be construed as falling within the scope of the present disclosure.

Hereinafter, the present disclosure will be described in more detail through Examples.

### EXPERIMENTAL EXAMPLE

### Experimental Example 1. Confirmation of Increase in NF1 Protein (GRD1 Domain) Through Nano-GloHiBiT Lytic Assay

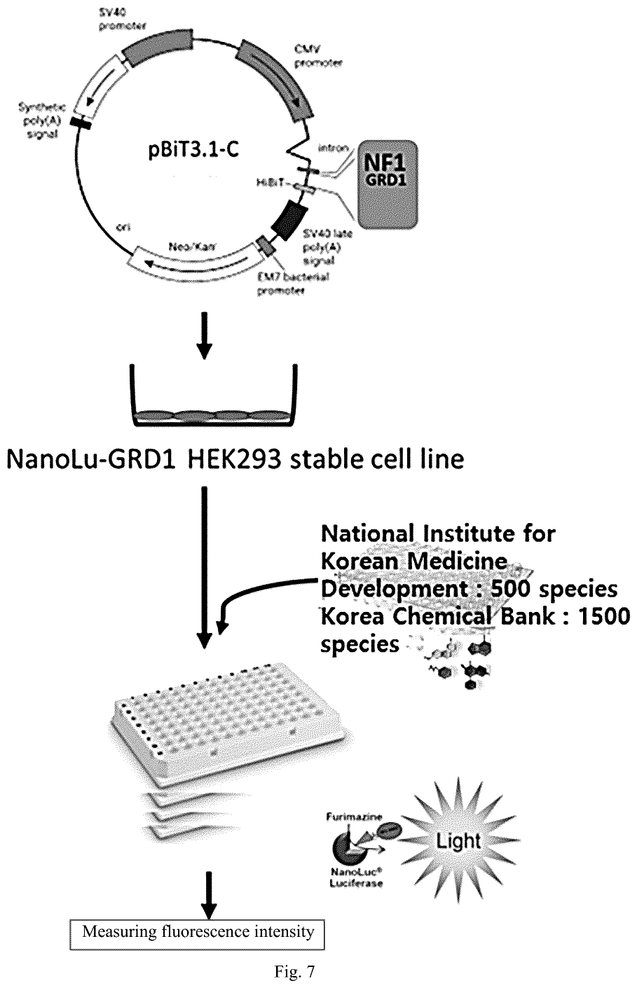

As illustrated in FIG. 7, NF1-GRD1 cDNA was loaded using a pBiT3.1-C vector conjugated with a 19 kDa Nanoluciferase reporter and then transfected into 293T cells to produce 293T cells (stable cell line) stably expressing the NF1-GRD1 protein.

Thereafter, the 293T cells were treated with a total of 2,000 natural products distributed from the National Institute for Korean Medicine Development (NIKOM) and Korea Chemical Bank for 24 hours, respectively, and then NF1-GRD1 was quantitatively measured using luciferase activity (Nano-Glo Hibit lytic assay) using a Nano-Glo (promega) sample, and substances with higher activity than a DMSO-treated control group were analyzed.

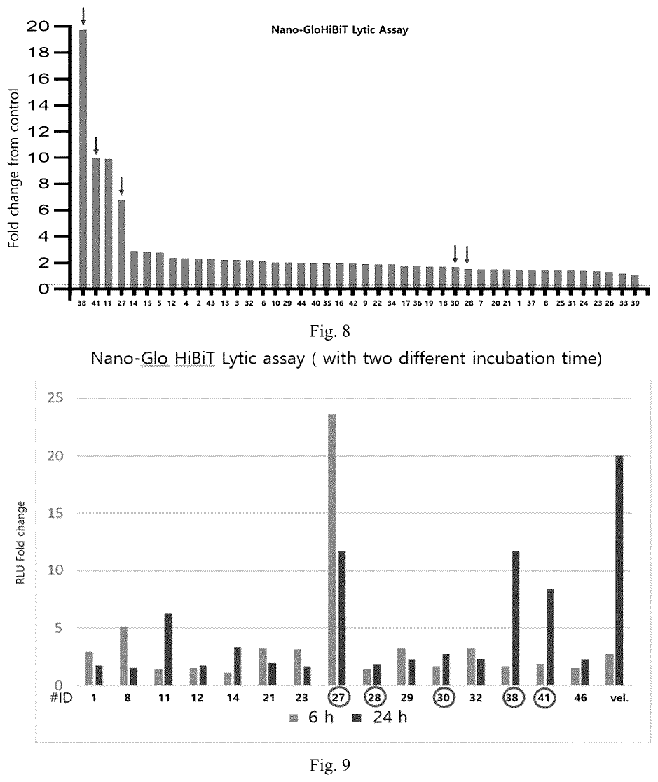

The results were illustrated in FIG. 8 below. Through screening, the present inventors discovered a total of 46 natural products that increased neurofibromin protein (NF1 protein). Among the natural products, as illustrated in FIG. 4, 27 refers to 20-O-acetylingenol-3-angelate (PN-NF27), 28 refers to (10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate (PN-NF28), 30 refers to R(−) Apomorphine hydrochloride hemihydrate (PN-NF30), 38 refers to Thiostrepton (PN-NF38), and 41 refers to Vorinostat (PN-NF41).

As illustrated in FIG. 8, PN-NF27 showed an effect of increasing NF1 protein about 6 times higher than a control group, PN-NF28 and PN-NF30 showed an effect of increasing NF1 protein about 2 times higher than the control group, and PN-NF38 showed an effect of increasing NF1 protein about 20 times higher than the control group, and PN-NF41 showed an effect of increasing NF1 protein about 10 times higher than the control group. That is, the natural products PN-NF27, PN-NF28, PN-NF30, PN-NF38 and PN-NF41 according to the present disclosure showed a significantly higher effect of increasing NF1 protein than the control group. As such, the natural products according to the present disclosure have the effect of improving the symptoms of NF1 haploinsufficiency, which occurs due to a quantitative decrease in functional NF1 protein (neurofibromin) by inducing a quantitative increase in NF1 protein.

### Experimental Example 2. Analysis of Effect of Increasing NF1 Protein According to Treatment Time

The cells were treated in the same manner as in Experimental Example 1 except that the treatment time was 6 hours and 24 hours, respectively, and the results of measuring the activity in each case were illustrated in FIG. 9.

As illustrated in FIG. 9, PN-NF27 showed the maximum effect within 6 hours (24-fold effect compared to the control group), and PN-NF38 and PN-NF41 showed the maximum effect at 24 hours (in the case of PN-NF38, 11-fold effect compared to the control group, and in the case of PN-NF41, 8-fold effect compared to the control group).

### Experimental Example 3. Analysis of Effect of Increasing NF1 Protein Through Western Blot

The effect of increasing NF1 protein in human renal epithelial-like cells (293T), human lung cancer cells (A549), and mouse embryonic fibroblasts (MEF) was analyzed using Western blot. Specifically, the cells incubated in a 10% FBS+DMEM culture medium for 48 hours were treated with 5 μM each of compounds for 6 hours, and then the quantified protein samples were subjected to electrophoresis. After electrophoresis, the proteins in the gel were transferred to a PVDF membrane and then Western blot was performed using NF1 antibody (Abcam, Cat#ab128054).

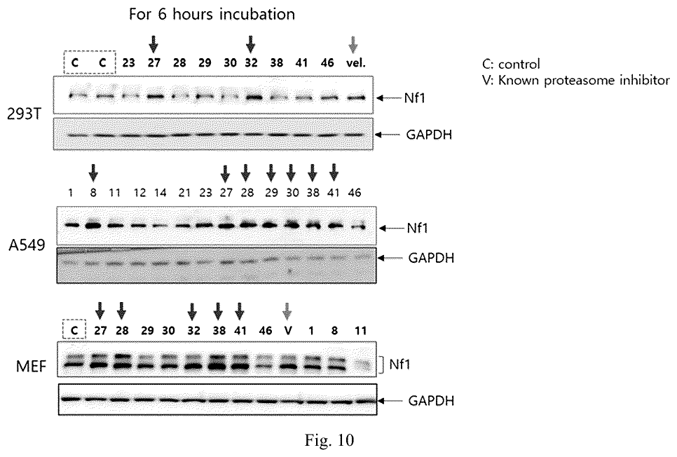

The results were illustrated in FIG. 10 below. In 293T cells, the NF1 protein increasing effect of PN-NF27 was excellent, and in A549 cells, the NF1 protein increasing effect of PN-NF27, PN-NF28, PN-NF30, PN-NF38, and PN-NF41 was superior to other substances. In addition, in MEF cells, the NF1 protein increasing effect of PN-NF27, PN-NF28, PN-NF38, and PN-NF41 was superior to other substances.

### Experimental Example 4. MTT Assay

Human lung cancer cells A549 and A549GR were treated with PN-NF27, PN-NF28, PN-NF30, PN-NF38, and PN-NF41, respectively, and then the MTT assay results were shown. Specifically, A549 and A549GR (anticancer resistant) cells incubated for 24 hours in a 96 well plate were treated with various concentrations of compounds for 72 hours, respectively, and then added with 10 μl of 3-(4,5-dimethylthiazole-2-yl)-2,5-diphenyltetrazolium bromide (MTT) and after 2 hours, the absorbance was measured at 550 nm.

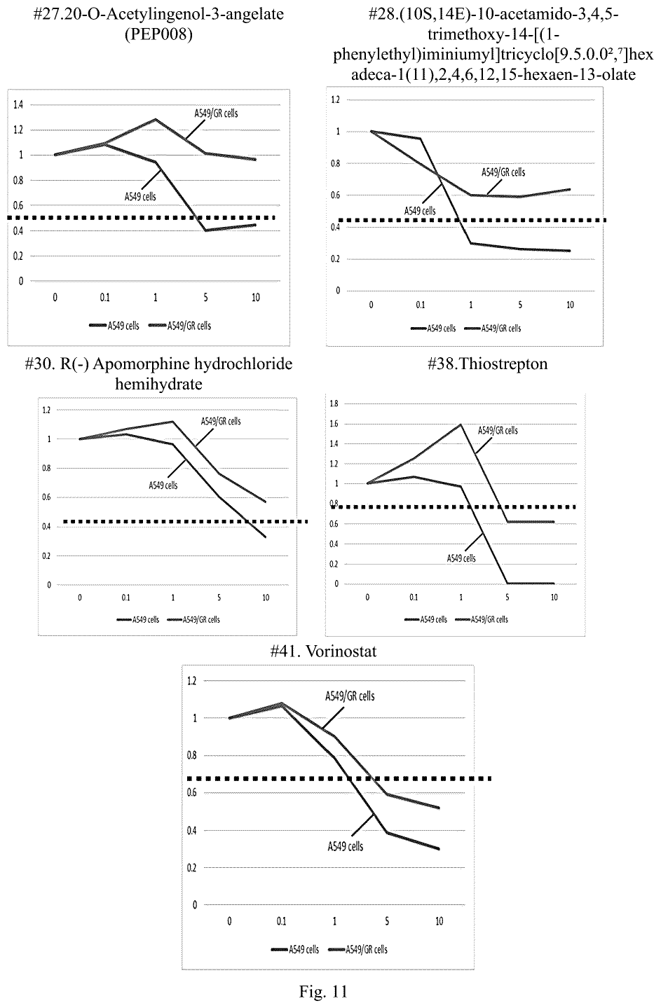

The results were illustrated in FIG. 11 below. Through FIG. 11, the effect of these compounds on cell proliferation of human lung cancer cells A549 and A549GR (anticancer resistance) may be seen. PN-NF27, PN-NF28, PN-NF30, PN-NF38, and PN-NF41 effectively inhibited cell proliferation of A549 and A549GR.

### Experimental Example 5. Effect Analysis of PN-NF27

### 5-1. Analysis of Increased NF1-GRD1 Protein According to Concentration and Time Using Nano-Glo HiBit Assay

The cells were treated in the same manner as in Experimental Example 1, except that the treatment time was 6 hours and 24 hours, respectively, and the concentration of PN-NF27 was 0 μM, 0.25 μM, 0.5 μM, 1.0 μM, and 5.0 μM, and the results of measuring the activity in each case were illustrated in FIG. 8.

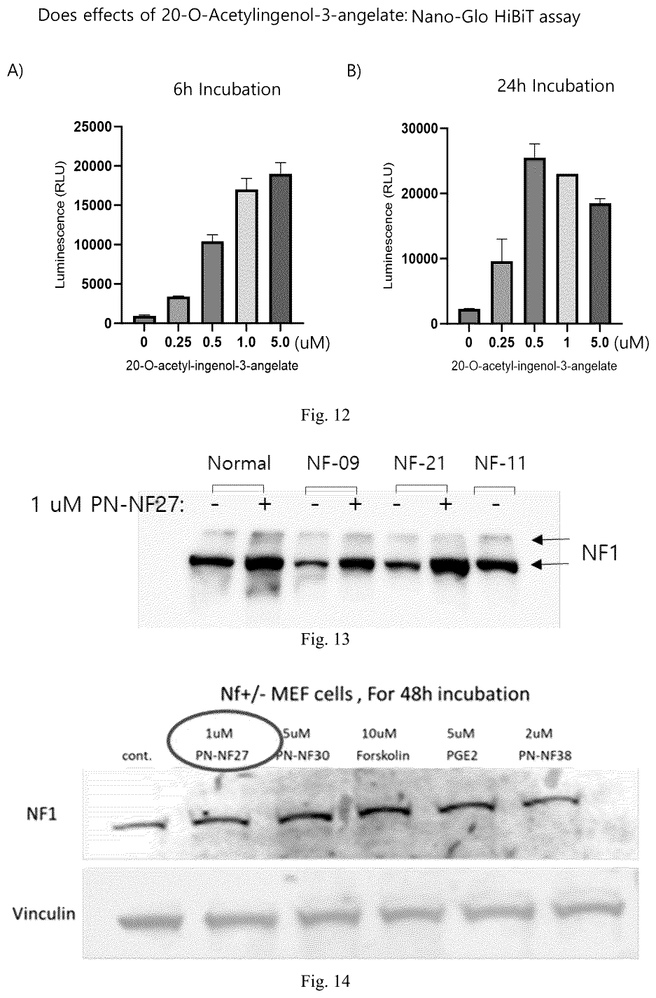

As illustrated in FIG. 12, the case treated with PN-NF27 showed higher activity than the case where PN-NF27 was not treated (0 μM). In particular, the highest activity was shown at 5.0 μM when treated for 6 hours and at 0.5 μM when incubated for 24 hours.

### 5-2. Analysis of Effect of Increasing NF1 Protein Using Western Blot

The effect of increasing NF1 protein in skin cells NF-09, NF-21, and NF-11 of NF1 patients was analyzed using Western blot. A specific method was as described in Experimental Example 3.

As a result, in both normal cells and NF-09, NF-21, and NF-11 cells, the NF1 protein tended to increase when treated with 1 μM of PN-NF27 compared to when not treated with 1 μM of PN-NF27 (see FIG. 13).

In addition, the effect of increasing NF1 protein in mouse embryonic fibroblasts (MEF) was analyzed using Western blot. A specific method was as described in Experimental Example 3.

As a result, the NF1 protein quantification tended to increase when treated with 1 μM of PN-NF27 compared to a control group (cont.) (see FIG. 14).

### 5-3. Cell Proliferation Inhibitory Effect of PN-NF27

The cell proliferation inhibitory effect of PN-NF27 was confirmed by an MTT method using skin cells NF-09, NF-21, and NF-11 of NF1 patients. Specifically, fibroblasts (passage>8) incubated from NF1 patient skin tissue were treated with 5 μM of PN-NF27 for 72 hours, and then added with 3-(4,5-dimethylthiazol-2-yl)-2,5-diphenyltetrazolium bromide (MTT) and reacted for 2 hours, and then the absorbance was measured at 550 nm.

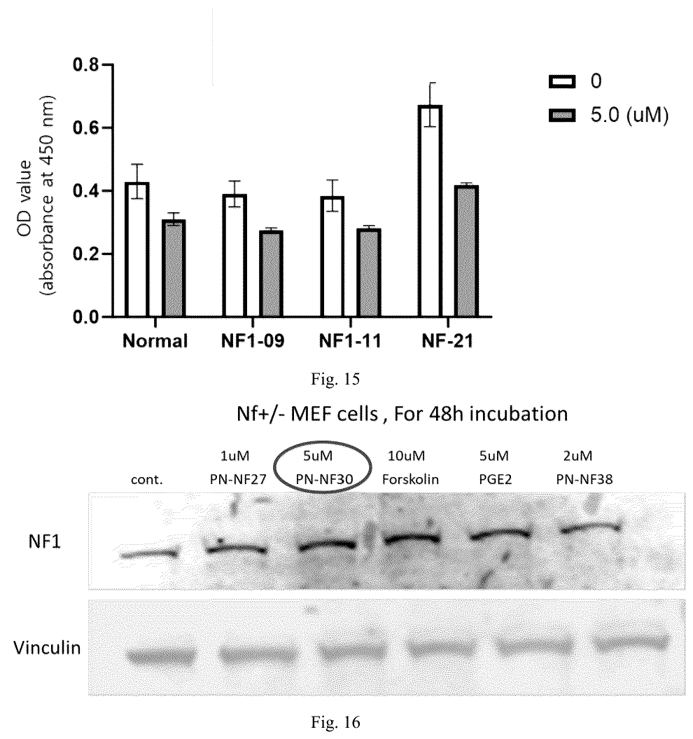

The results were illustrated in FIG. 15 below. As a result, in both normal cells and NF-09, NF-21, and NF-11 cells, the cell proliferation tended to be inhibited when treated with 1 μM of PN-NF27 compared to when not treated with 1 μM of PN-NF27. As such, since PN-NF27 has the effect of inhibiting cell proliferation and increasing NF1 protein, it is expected to be used as a cancer therapeutic agent.

### Experimental Example 6. Effect Analysis of PN-NF30

### 6-1. Analysis of Effect of Increasing NF1 Protein Using Western Blot

The effect of increasing NF1 protein in mouse embryonic fibroblasts (MEF) was analyzed using Western blot. A specific method was as described in Experimental Example 3.

As a result, the NF1 protein quantification tended to increase when treated with 5 μM of PN-NF30 compared to a control group (cont.) (see FIG. 16).

### 6-2. Cell Proliferation Inhibitory Effect

The cell proliferation inhibitory effect of PN-NF30 was confirmed using human colon cancer cells HCT-8, HCT-15, and HCT-116, human pancreatic cancer cells PANC1, and human prostate cancer cells PC3. Specifically, the HCT-8, HCT-15, HCT-116, PANC1, and PC3 cells incubated for 24 hours in a 96 well plate were treated with compounds at concentrations of 0, 0.1, 0.5, 1, 5, and 10 μM for 72 hours, respectively, and then added with 10 μl of 3-(4,5-dimethylthiazole-2-yl)-2,5-diphenyltetrazolium bromide (MTT) and after 1 hours, the absorbance was measured at 550 nm.

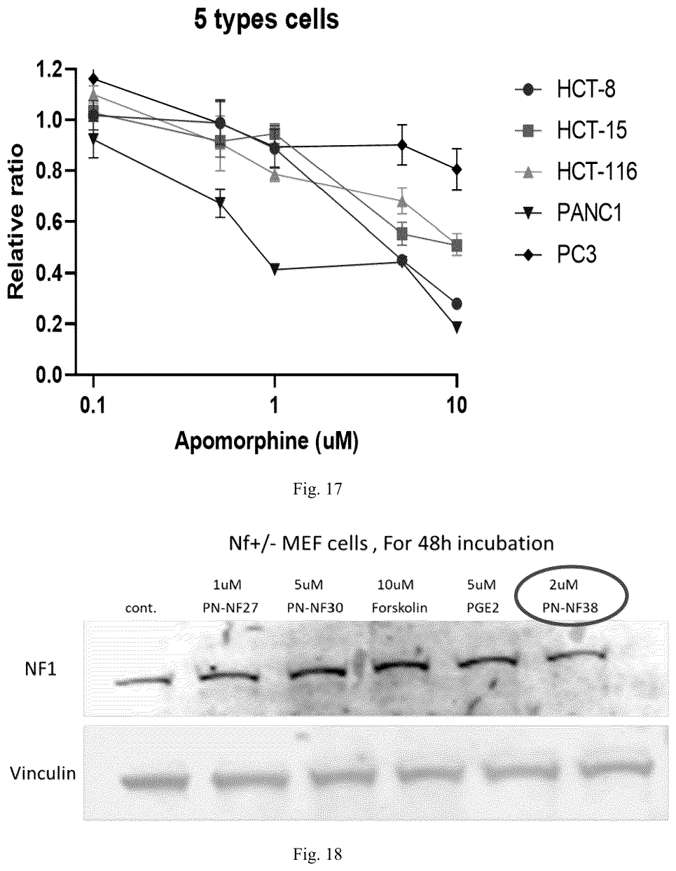

As a result, PN-NF30 tended to effectively inhibit cell proliferation as the concentration increased (see FIG. 17).

### Experimental Example 7. Effect Analysis of PN-NF38

### 7-1. NF1 Protein Increasing Effect

The effect of increasing NF1 protein in mouse embryonic fibroblasts (MEF) was analyzed using Western blot. A specific method was as described in Experimental Example 3.

As a result, the NF1 protein quantification tended to increase when treated with 2 μM of PN-NF38 compared to a control group (cont.) (see FIG. 18).

### 7-2. Cell Proliferation Inhibitory Effect

The cell proliferation inhibitory effect of PN-NF38 was confirmed using skin cells NF-09, NF-21, and NF-11 of NF1 patients. Specifically, skin fibroblasts (passage>8) incubated for 48 hours were treated with PN-NF38 at 0, 0.1, 1, 2.5, 5, and 10 μM for 24 hours, and then added with 3-(4,5-dimethylthiazol-2-yl)-2,5-diphenyltetrazolium bromide (MTT) and reacted for 1 hours, and then the absorbance was measured at 550 nm.

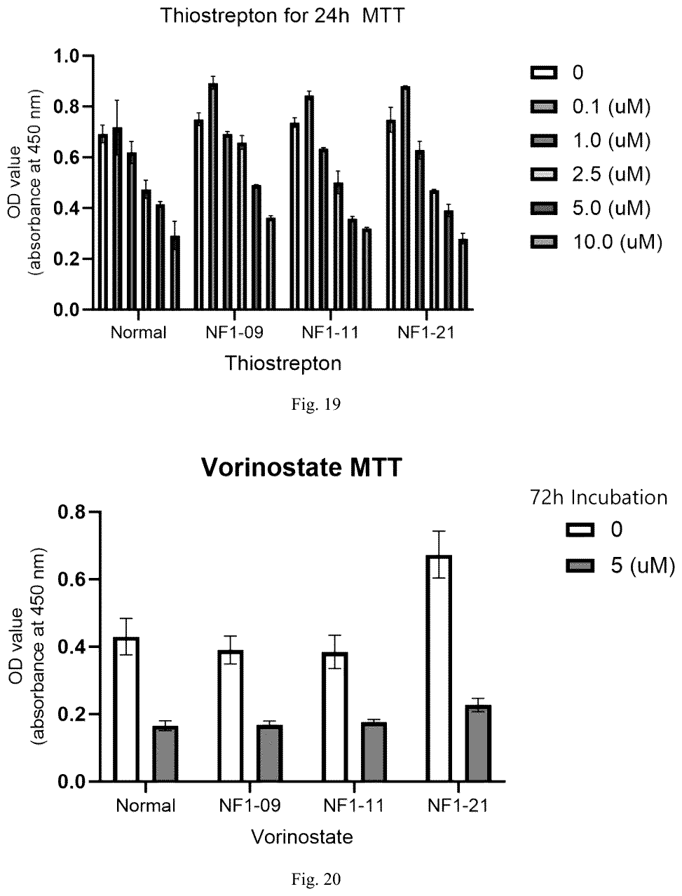

As a result, cell proliferation tended to be effectively inhibited as the concentration of PN-NF38 cells increased. In particular, at 10 μM, the cell viability was only less than 0.3 (see FIG. 19). As such, since PN-NF38 has the effect of inhibiting cell proliferation and protein degradation, it is expected to be used as a cancer therapeutic agent and a therapeutic agent for multiple myeloma and lymphoma.

### Experimental Example 8. Cell Proliferation Inhibitory Effect of PN-NF41

The cell proliferation inhibitory effect of PN-NF41 was confirmed using skin cells NF-09, NF-21, and NF-11 of NF1 patients. Specifically, skin fibroblasts (passage>8) incubated for 24 hours were treated with 5 μM of PN-NF41 for 72 hours, and then added with 3-(4,5-dimethylthiazol-2-yl)-2,5-diphenyltetrazolium bromide (MTT) and reacted for 1 hours, and then the absorbance was measured at 550 nm.

The results were illustrated in FIG. 20 below. In both normal cells and NF-09, NF-21, and NF-11 cells, the cell proliferation tended to be inhibited when treated with 5 μM of PN-NF41 compared to when not treated with 5 μM of PN-NF41.

As such, since the natural compounds PN-NF27, PN-NF28, PN-NF30, PN-NF38, and PN-NF41 of the present disclosure show excellent effects on inducing the increase in NF1 protein, inhibiting the protein degradation, and inhibiting the cell proliferation compared to the control group, the natural compounds may be usefully used as various applications including a therapeutic agent for neurofibromatosis type 1, an anticancer agent, and a therapeutic agent for multiple myeloma and lymphoma.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A pharmaceutical composition, comprising at least one compound(s) or pharmaceutically acceptable salt thereof selected from the group consisting of:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

2. The pharmaceutical composition of claim 1, wherein the concentration of the 20-O-acetylingenol-3-angelate is 0.1 to 10 μM.

3. The pharmaceutical composition of claim 1, wherein the concentration of the (10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate is 0.1 to 10 μM.

4. The pharmaceutical composition of claim 1, wherein the concentration of the R(−) Apomorphine hydrochloride hemihydrate is 1.0 to 10 μM.

5. The pharmaceutical composition of claim 1, wherein the concentration of the Thiostrepton is 1.0 to 10 μM.

6. The pharmaceutical composition of claim 1, wherein the concentration of the Vorinostat is 1.0 to 10 μM.

7. The pharmaceutical composition of claim 1, wherein the pharmaceutical composition induces an increase in neurofibromin protein (NF1 protein).

8. A method for treating neurofibromatosis type 1 comprising (a) administering into a subject in need thereof at least one compound(s) selected from the group consisting of:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

9. The method for treating neurofibromatosis type 1 of claim 8, wherein the subject is a patient with neurofibromatosis type 1.

10. A pharmaceutical composition for preventing or treating cancer, comprising at least one compound(s) or pharmaceutically acceptable salt thereof selected from the group consisting of:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6,12,15- hexaen-13-olate;
R(−) Apomorphine hydrochloride hemihydrate;
Thiostrepton; and
Vorinostat.

11. The pharmaceutical composition of claim 10, wherein the cancer is gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer or bone marrow tumor.

12. The pharmaceutical composition of claim 11, wherein the blood cancer is multiple myeloma or lymphoma.

13. A method for preventing or treating cancer comprising (a) administering to a patient in need thereof at least one compound(s) selected from the group consisting of:
20-O-acetylingenol-3-angelate;
(10S,14E)-10-acetamido-3,4,5-trimethoxy-14-[(1-phenylethyl)iminiumyl]tricyclo[9.5.0.0^{2,}^{⁷}]hexadeca-1(11),2,4,6, 12,15-hexaen-13-olate;
R(−) Apomorphine hydrochloride hemihydrate;
Thiostrepton; and
Vorinostat.
